(19) United States
(12) Patent Application Publication
(10) Pub. No.: US 2013/0165835 A1
(43) Pub. Date: Jun. 27, 2013

(54) BANDAGES

(71) Applicant: Richard A. Lewis, Chicago, IL (US)

(72) Inventor: Richard A. Lewis, Chicago, IL (US)

(21) Appl. No.: 13/726,863

(22) Filed: Dec. 26, 2012

Related U.S. Application Data
(60) Provisional application No. 61/579,547, filed on Dec. 22, 2011.

(51) Int. Cl. A61F 13/00

(52) U.S. Cl. CPC A61F 13/00021
USPC 602/43

(57) ABSTRACT

A skin guard apparatus for protecting a part of an anatomy is provided herein. The apparatus includes a covering member configured to correspond to the part of the anatomy, wherein the covering member does not touch the part of the anatomy, a raiser member, the raiser member configured to raise the covering member off the part of the anatomy, and one or more positioning members configured for the part of the anatomy.

### CROSS-REFERENCE TO RELATED APPLICATIONS

This application claims the benefit of U.S. Provisional Application 61/579,547 filed on Dec. 22, 2011, the entirety of which is incorporated herein by reference.

### BACKGROUND

Skin protection devices are commonly used to cover up burns, scrapes, bruises, skin grafts and other injuries of the skin. Many devices that are currently available are inadequate in that they do not allow for airflow, do not provide enough physical protection and do not function well in a wearer's every day life commitments and work. Most also require adhesive tape, in order to be affixed to skin, and only work on flat surfaces. Overall, currently, the available products are devices that gets dirty, soggy, or even infected. Most current injury protection systems utilize absorbent fibrous materials, sometimes encased in a membranous film, and attached with an adhesive tape. There are many disadvantages. Furthermore, the close proximity to the injury site and non structural nature of the currently available devices allow for susceptibility to irritation and/or additional injury to an existing injury.

### SUMMARY

Bandages are disclosed herein. According to one aspect, the bandages provide structural shielding to protect an injury site from external accidental irritation. The bandages isolate the injury site for the purpose of restricting infection to an open wound and for the application of antiseptic treatment. The bandages also reduce external sensation or irritation.

Depending upon the location of the injury, the injury itself, even with a bandage applied, can be too sensitive to allow for continued use during the healing process. According to one aspect of the disclosed technology, the bandages allow the wearer to extend the use of an injured body part. The bandages eliminate the accumulation of dirt, blood, bacteria, etc. on the bandage itself as a result of the fluid state of the antiseptic dressing, material texture, and adhesive residue at least because the replacement of the complete bandage is not necessary.

The bandages eliminate the need to utilize adhesives that create additional irritation of the area surrounding the injury site usually caused by the repeated removal/replacement of the tape used to hold the dressing in place. Restriction of airflow caused by the density of the fibrous material and the mostly impenetrable surface area of the adhesive tape creates a anaerobic environment inhibiting the healing process. However, the bandages eliminate such restriction.

The bandages are safe and reusable once saturated with water, allowing the wearer to have an improved quality of life. Finally, the bandages do not deteriorate with use, and may be easily and repeatedly removed or repositioned. Attachment mechanisms that are part of the bandages do not deteriorate. The wearer need no replace the bandages often due to deterioration of the surface or attachment mechanism. The bandages provide for an improved injury guard device that allows for air circulation around the injury site and provides a sturdy structure that shields the injury site from further impact, irritation, or unintentional contact.

As such, a skin guard apparatus for protecting a part of an anatomy is described herein. The apparatus comprises a covering member configured to correspond to the part of the anatomy, wherein the covering member does not touch the part of the anatomy, a raiser member, the raiser member configured to raise the covering member off the part of the anatomy, and one or more positioning members configured for the part of the anatomy.

The skin guard apparatus may include one or more of the following features. The apparatus may additionally include a fastener for securing the skin guard apparatus around the part of the anatomy. At least the covering member and the raiser member of the skin guard apparatus may be made of a rigid or semi-rigid material. The raiser member may be attached to the covering member. The raiser member may sit atop the part of the anatomy. The raiser member may be continuous with the covering member.

The one or more positioning members may be configured to hold the covering member in place atop the part of the anatomy. The one or more positioning members may be configured to hold the covering member in place atop the part of the anatomy by wrapping around the circumference of the part of the anatomy. The covering member may include one or more sub-members.

According to another aspect of the disclosed technology, a covering member may be configured to correspond to the part of the anatomy, wherein the covering member has a curve that creates a dome over the part of the anatomy and wherein the covering member does not touch the part of the anatomy. The raiser member may be a part of the covering member, as defined by the curve of the covering member.

It is understood that other configurations of the subject technology will become readily apparent from the following detailed description, where various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

Certain features of the subject technology are set forth in the appended claims. However, for purpose of explanation, several implementations of the subject technology are set forth in the following figures.

FIG. 1 illustrates an example skin guard apparatus for protecting a finger.

FIG. 2 illustrates an example skin guard apparatus 200 for protecting a thumb.

FIG. 3 illustrates an example skin guard apparatus 300 for protecting a knuckle.

FIG. 4 illustrates an example skin guard apparatus for protecting a top of a hand.

FIG. 5 illustrates example skin guard apparatus for protecting a top of a hand.

FIG. 6 illustrates an example skin guard apparatus for protecting a palm of a hand.

FIG. 7 illustrates an example skin guard apparatus for protecting a palm of a hand.

FIG. 8 illustrates an example skin guard apparatus for protecting an elbow.

FIG. 9 illustrates an example skin guard apparatus for protecting a knee.

### DETAILED DESCRIPTION

In the following detailed description, numerous specific details are set forth to provide a full understanding of the present disclosure. It will be apparent, however, to one ordinarily skilled in the art that the configurations of the present disclosure may be practiced without some of these specific details. In other instances, well-known structures and techniques have not been shown in detail so as not to obscure the disclosure.

According to an aspect of the disclosed technology, the bandages enhance the mobility as well as the utility of injured parts of the anatomy or areas of skin. The bandages help eliminate pain from accidental contact with injury site and help to isolate injury site from any physical contact. The bandages help transfer external pressure and/or physical contact to non sensitive area(s), as well as promote air flow, due to the raiser members. Thus, the bandages provide complete air circulation to injury site for enhanced aerobic healing. The bandages may be used alone or in combination with traditional medicinal dressings.

The bandages are easy to keep clean and are washable. They are preferably made out of medical grade plastic, rigid or semi-rigid similar material or other suitable material. They may be made out of an anti-bacterial material. It is preferable the material is non-porous, so as to resist dirt, stain, and bacteria growth.

According to one aspect, the bandages are made of a waterproof material that makes the bandages safe to use in the bath or shower, during swimming or other activities involving water.

The bandages may be adjustable. A hoof and loop type attachment may be used to secure the bandage. Velcro may be used, belt/buckle, strap, band mechanisms or other suitable closures or fasteners. Bandages are effortless to position, adjust, attach and remove.

A different shape and style of bandage may be used for different parts of the anatomy. For example, a finger bandage may be different from a thumb bandage and from an elbow bandage. Configurations may include bandages shaped specifically for feet, elbows, fingers, palm (inside), outside hand, arm, leg, etc. Thus, the bandages are able to conform ergonomically to the part of the anatomy, with minimum contact. The shape of the bandage is such that, the injury site is not in contact with the bandage, or is in minimal contact with one or more of the positioning members that attach the bandage to the part of the anatomy.

The bandage may be flexible and may bend easily with the natural movement of a particular part of the anatomy. The covering member may, therefore, be made out of several sub-members that allow the flexibility and movement.

The bandages are sleek and thin, and may be worn over or under clothing. According to one aspect, the bandages may be 0.1 mm-5 mm thick, depending on the anatomy part for which they are suited. It may be preferable that, bandages configured for fingers are thinner than bandages configured for feet or elbows, for example. Likewise, it may be preferable that, bandages configured for a leg are thicker than bandages configured for a palm, when the wearer needs to utilize the hand. Various sizes, thicknesses and shapes may be accommodated by the bandages described herein. Sensory pads may be added to bandages configured for fingers, for example, to enable the wearer to utilize modern touch screen devices or other devices sensitive to the type of material that touches them, for operation.

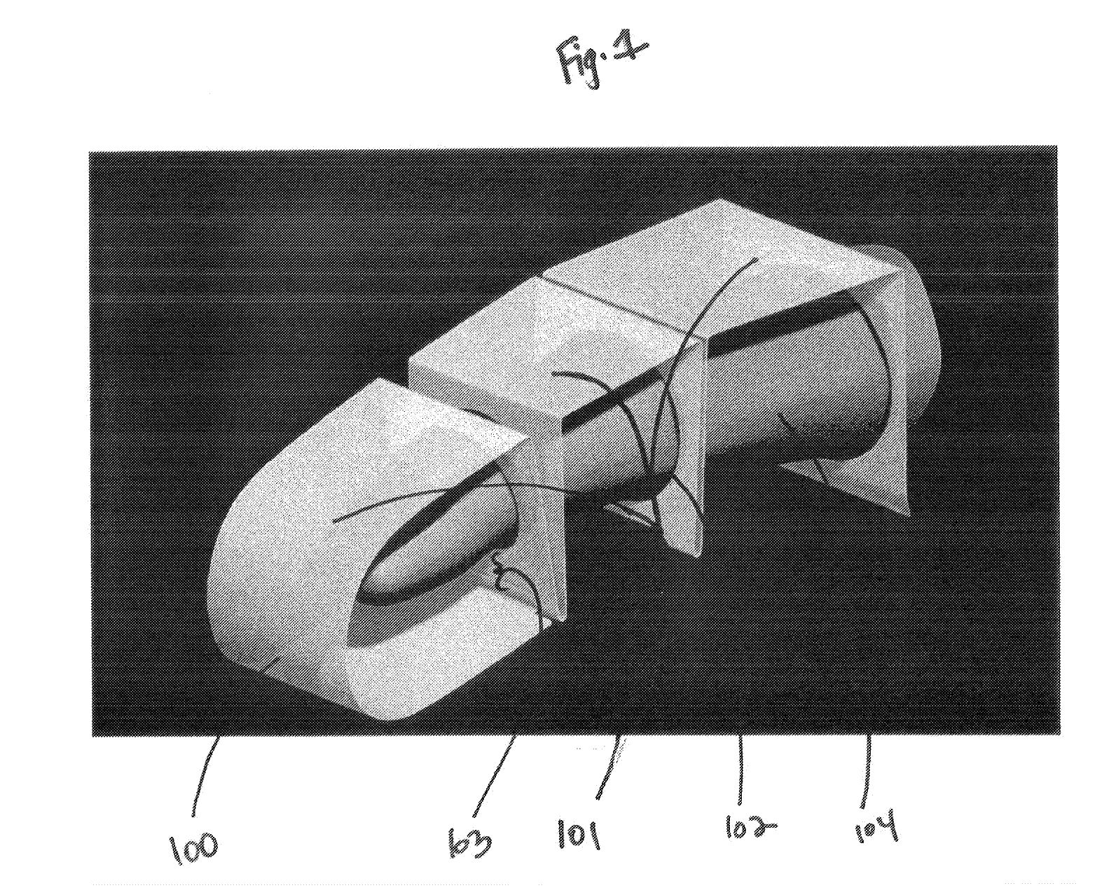

FIG. 1 illustrates an example skin guard apparatus 100 for protecting a finger. The example skin guard apparatus 100 is configured for a finger 104 anatomy part. The apparatus 100 includes a plurality of covering members 100 configured to correspond to the part of the anatomy (finger 104). The covering members 101 do not touch the finger 104. The apparatus 100 also includes a plurality of raiser members 103. The raiser members 103 raise the covering members off the finger 104. A raiser member 103 may be continuous with the covering member 101, as illustrated herein. Alternatively, a raising member 103 may be a separate structure The skin guard apparatus 100 also includes a plurality of positioning members 102 that are configured for the finger 104. The positioning members 102 depend on the type of the anatomy part for which a skin guard apparatus is intended. For example, for a finger, a circular structure through which the finger may be inserted may be used, as illustrated in FIG. 1.

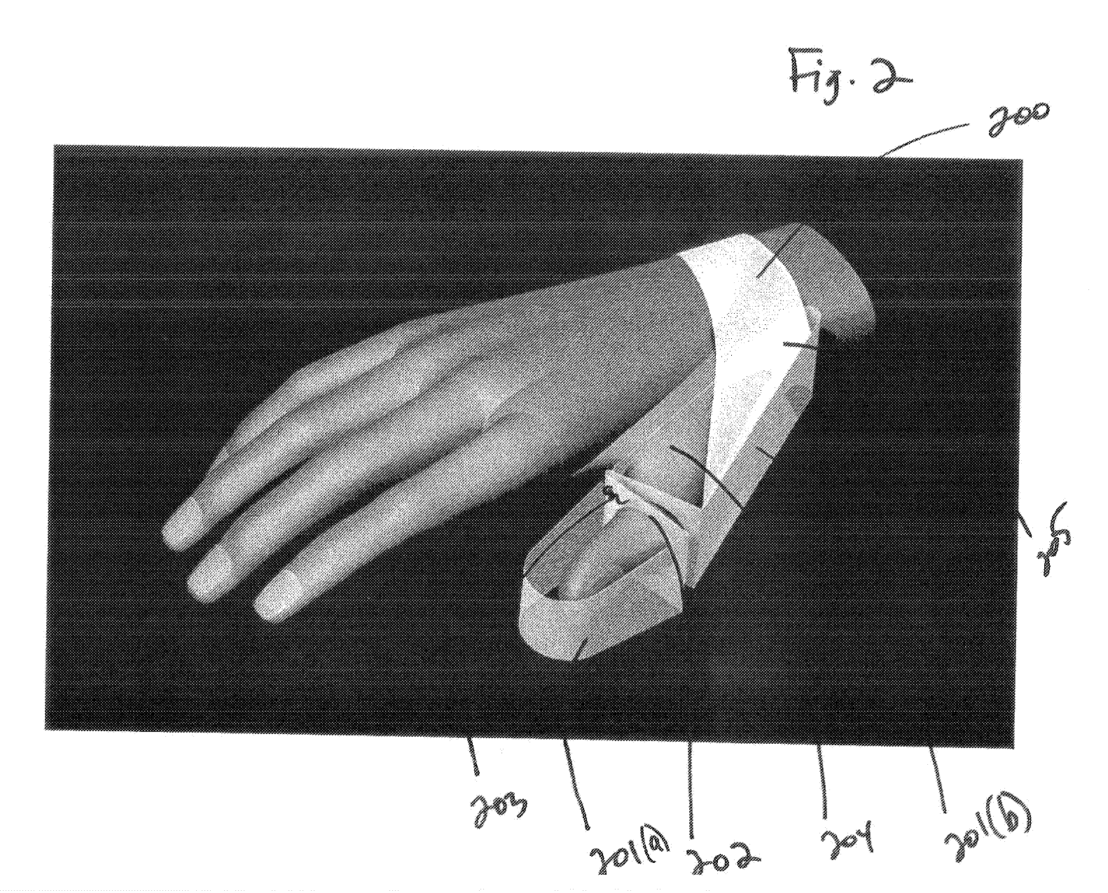

FIG. 2 illustrates an example skin guard apparatus 200 for protecting a thumb. The example skin guard apparatus 200 is configured for a thumb 204 anatomy part. The apparatus 200 includes a plurality of covering members 201, a raiser member 203 and positioning members 202. The apparatus 200 is configured from two segments, each having a covering member 201, a positioning member 202 and a plurality of raising members 203. The raising members 203 are continues, that is, they flow from the positioning members 202, on all sides. The apparatus 200 also has a fastener 205 for securing the skin guard apparatus around the part of the anatomy. The fastener 205 may be attached to any portion of the apparatus. As illustrated in FIG. 2, the fastener is attached to, or extends from the covering member 201(b).

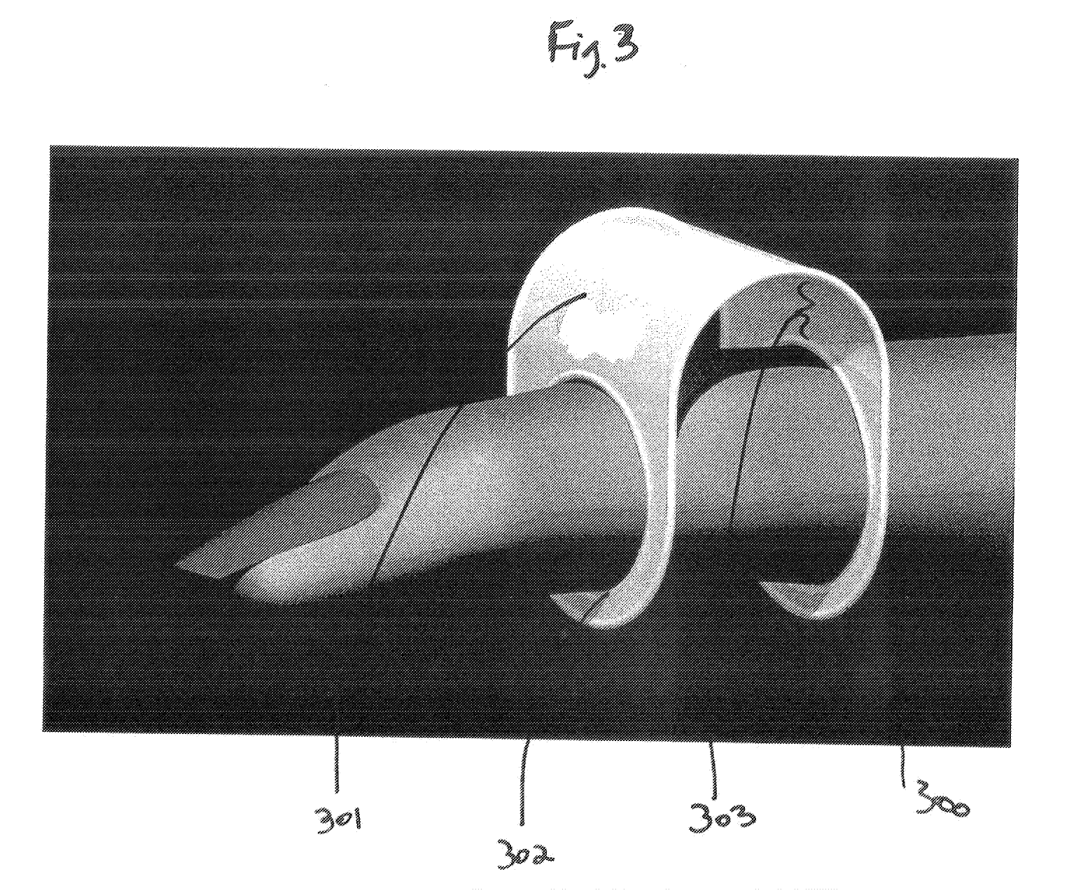

FIG. 3 illustrates an example skin guard apparatus 300 for protecting a knuckle. Apparatus 300 illustrates a covering member 301 that is curved and shaped like a dome. The apparatus 300 has two positioning members 302, that are circular, for a finger to be inserted therein. The raiser member 303 is merged with or extends from the covering member 301, and is defined by the curve of the covering member 301.

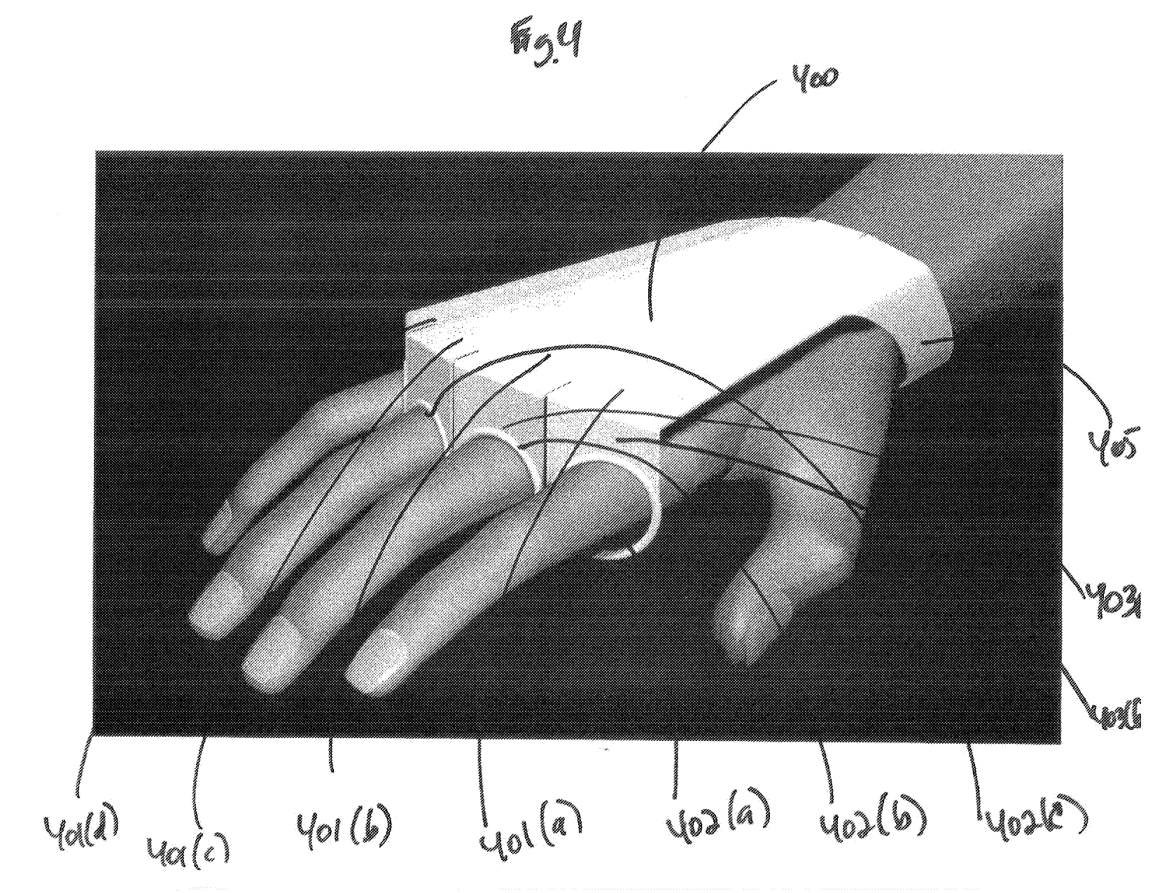

FIG. 4 illustrates an example skin guard apparatus for protecting a top of a hand. The skin guard apparatus 400 includes a plurality of covering members 401 (401 a,b,c,d). The plurality of covering members as illustrated herein may be referred to as sub-members of a covering member 401. As illustrated in FIG. 4, each sub-member of covering member 401, or each covering member 401, if each sub-member is treated separately, corresponds with a positioning member 402. That is, it may be implemented that each covering member has a separate positioning member. Also, a positioning member may be utilized by more than one covering member. The covering sub-members may be attached to each other or partially attached to each other via other members of a particular apparatus, depending on implementation. Separation or partial separation of the covering sub-members may aid in the mobility of the part of the anatomy, for example, fingers. The raiser members 403 are the height between the positioning members 402 and the covering members 401. A fastener 405 attaches the apparatus to the hand.

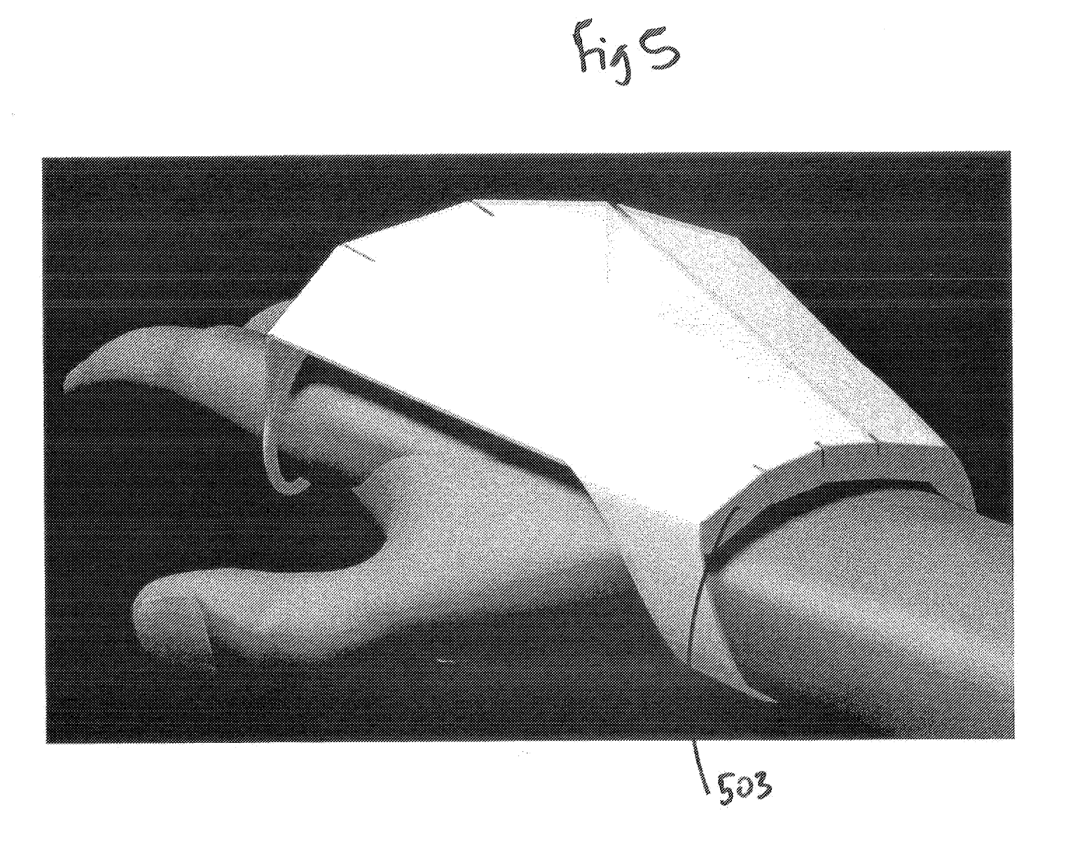

FIG. 5 illustrates example skin guard apparatus for protecting a top of a hand. It is the same apparatus as illustrated in FIG. 4, but from a different angle. From this depiction, raiser members 503 are visible. Thus, multiple raiser members may be utilized in an apparatus. The raiser members 503 are not attached to the positioning members. Instead, they are connected to the covering members, and are created by a right or semi-right angle. The height of the raisers may be anything from 2 mm-20 mm, depending on implementation.

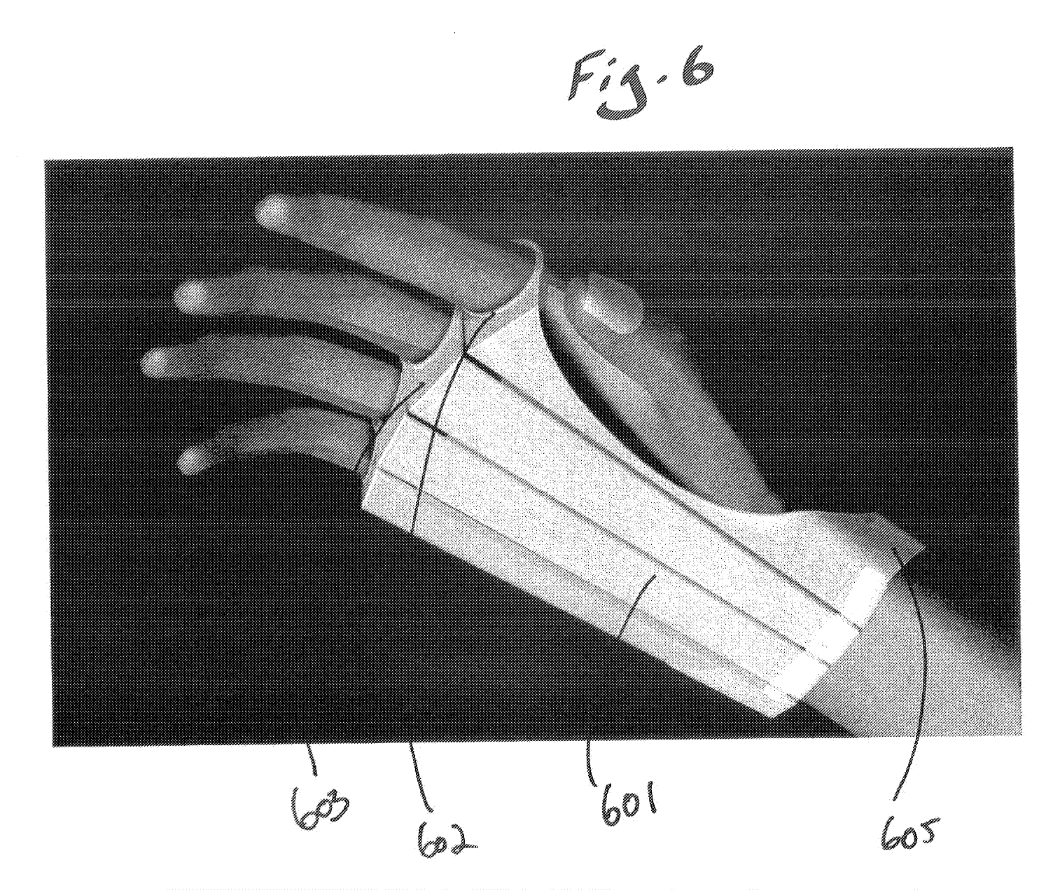

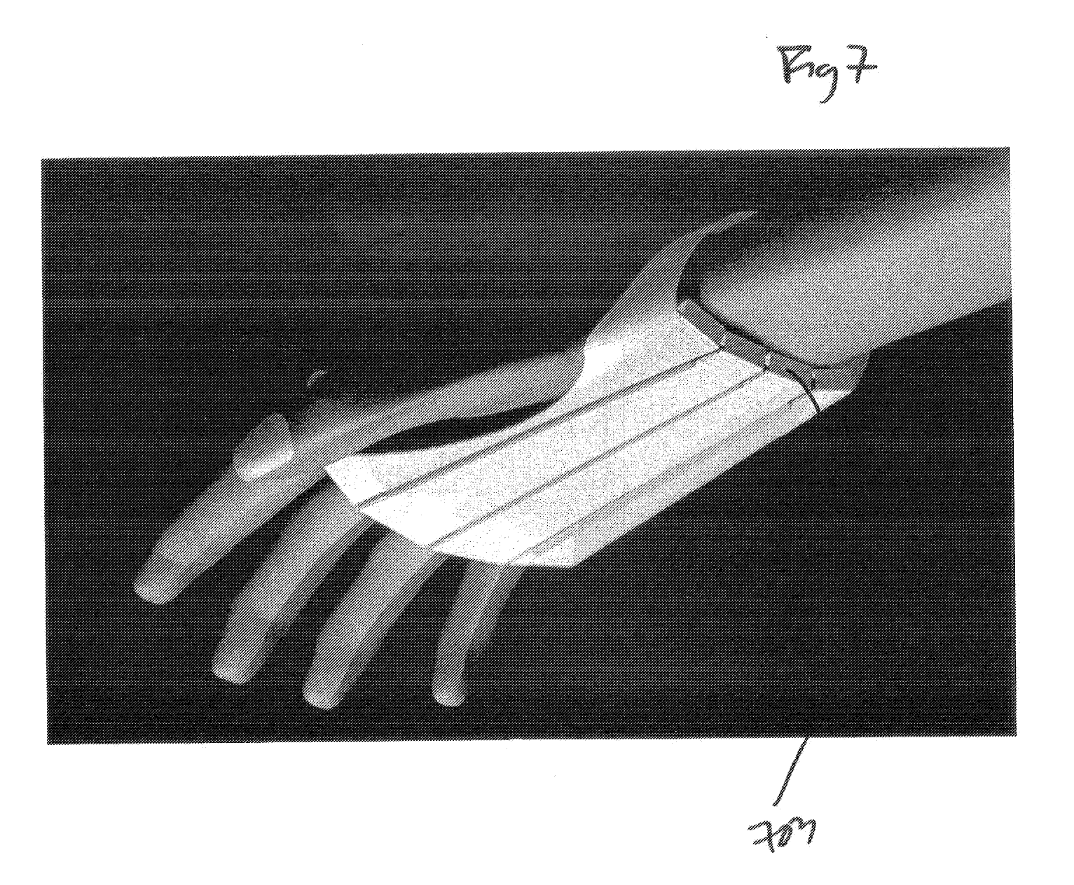

FIG. 6 illustrates an example skin guard apparatus for protecting a palm of a hand. The skin guard apparatus 600 includes a plurality of covering member 601, a plurality of positioning members 602 and a plurality of raiser members 603. FIG. 6 also depicts a fastener 605. FIG. 7 illustrates an example skin guard apparatus for protecting a palm of a hand. It shows additional raiser members 703 for the apparatus for protecting a palm of a hand.

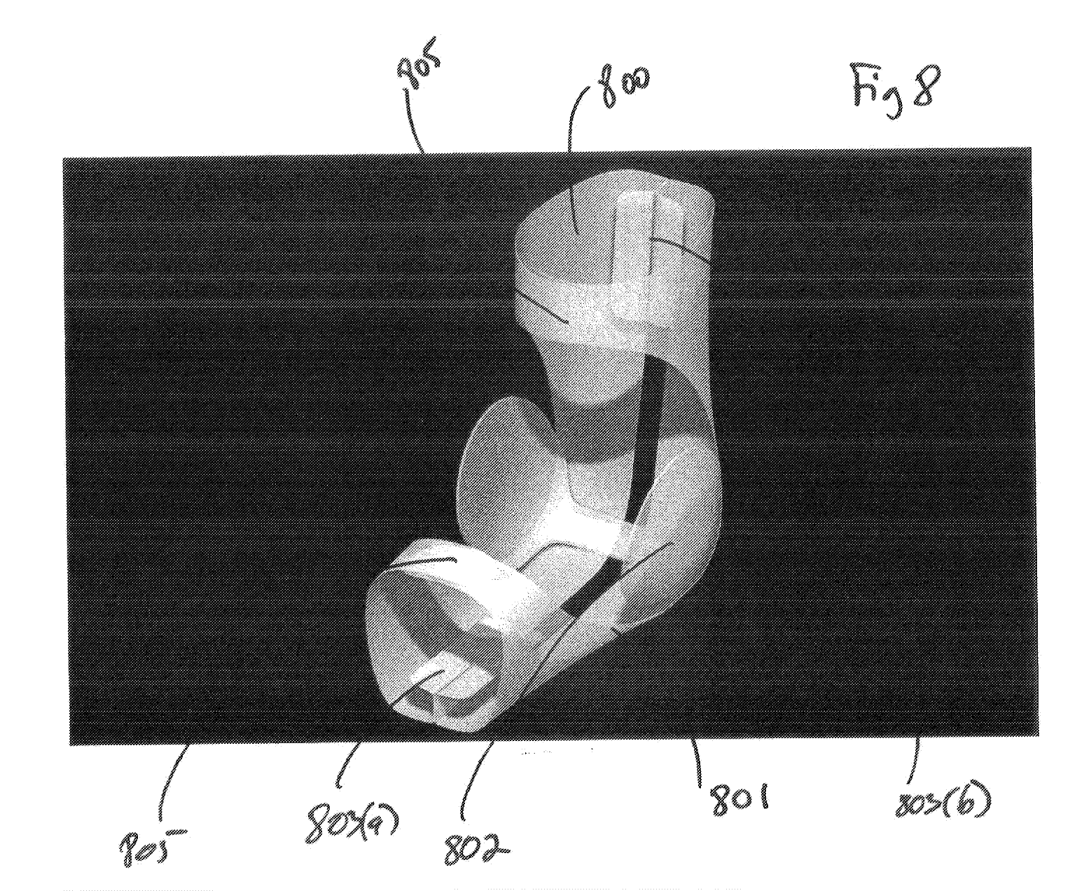

FIG. 8 illustrates an example skin guard apparatus for protecting an elbow. The apparatus 800 illustrates a covering member 801 that is configured to correspond to an elbow. The apparatus 800 includes positioning member 802 that help the apparatus attach to the elbow and raising members 803 that raise the apparatus. As illustrated herein, raising members 803 may be separate structures that are added to create a certain lift of the apparatus that creates a space between the part of the anatomy and the covering member. Fastener 805 helps keep the apparatus in place.

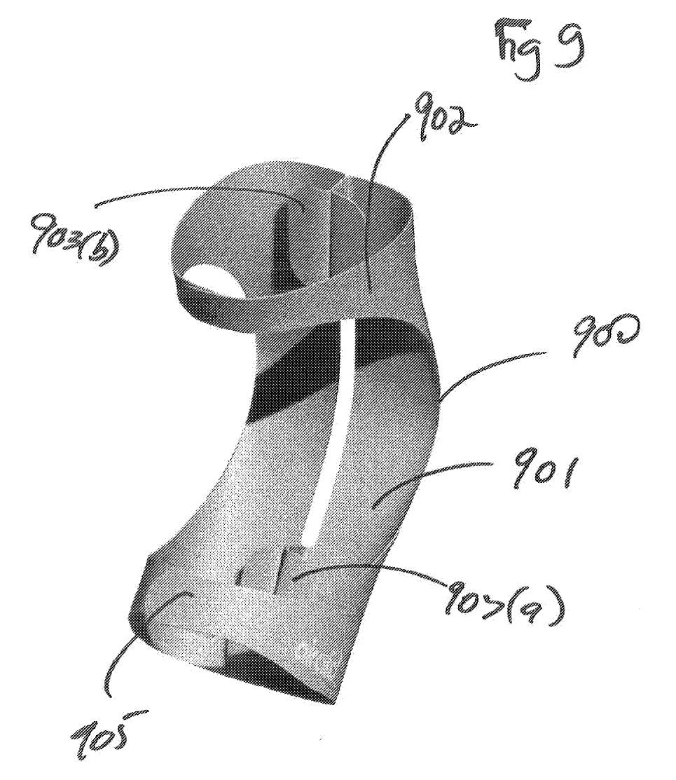

FIG. 9 illustrates an example skin guard apparatus for protecting a knee. The apparatus 900 illustrates a covering member 001 that is configured to correspond to a knee. The apparatus 900 includes positioning member 902 that help the apparatus attach to the knee and raising members 903 that raise the apparatus. As illustrated herein, raising members 903 may be separate structures that are added to create a certain lift of the apparatus that creates a space between the part of the anatomy and the covering member. Fastener 905 helps keep the apparatus in place.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the subject disclosure.

What is claimed is:

1. A skin guard apparatus for protecting a part of an anatomy, the apparatus comprising:
a covering member configured to correspond to the part of the anatomy, wherein the covering member does not touch the part of the anatomy;
a raiser member, the raiser member configured to raise the covering member off the part of the anatomy; and
one or more positioning members configured for the part of the anatomy.

2. The skin guard apparatus of claim 1, further comprising:
a fastener for securing the skin guard apparatus around the part of the anatomy.

3. The skin guard apparatus of claim 1, wherein at least the covering member and the raiser member are made of a rigid or semi-rigid material.

4. The skin guard apparatus of claim 1, wherein the raiser member is attached to the covering member.

5. The skin guard apparatus of claim 1, wherein the raiser member sits atop the part of the anatomy.

6. The skin guard apparatus of claim 1, wherein the raiser member is continuous with the covering member.

7. The skin guard apparatus of claim 1, wherein the one or more positioning members is configured to hold the covering member in place atop the part of the anatomy.

8. The skin guard apparatus of claim 1, wherein the one or more positioning members is configured to hold the covering member in place atop the part of the anatomy by wrapping around the circumference of the part of the anatomy.

9. The skin guard apparatus of claim 1, wherein the covering member comprises one or more sub-members.

10. A skin guard apparatus for protecting a part of an anatomy, the apparatus comprising:
a covering member configured to correspond to the part of the anatomy, wherein the covering member has a curve that creates a dome over the part of the anatomy and wherein the covering member does not touch the part of the anatomy;
a raiser member, the raiser member configured to raise the covering member off the part of the anatomy; and
one or more positioning members configured for the part of the anatomy.

11. The skin guard apparatus of claim 10, wherein the covering member is comprised of one or more sub-members.

12. The skin guard apparatus of claim 10, further comprising:
a fastener for securing the skin guard apparatus around the part of the anatomy.

13. The skin guard apparatus of claim 10, wherein at least the covering member and the raiser member are made of a rigid or semi-rigid material.

14. The skin guard apparatus of claim 10, wherein the raiser member is attached to the covering member.

15. The skin guard apparatus of claim 10, wherein the raiser member sits atop the part of the anatomy.

16. The skin guard apparatus of claim 10, wherein the one or more positioning members is configured to hold the covering member in place atop the part of the anatomy by wrapping around the circumference of the part of the anatomy.

17. The skin guard apparatus of claim 10, wherein the raiser member is continuous with the covering member.

18. The skin guard apparatus of claim 10, wherein the one or more positioning members is configured to hold the covering member in place atop the part of the anatomy.

19. The skin guard apparatus of claim 10, wherein the raiser member is a part of the covering member, as defined by the curve of the covering member.

20. The skin guard apparatus of claim 10, wherein the raiser member corresponds to a covering member, when there is more than one raiser member and more than one covering member.
